(19) United States
(12) Patent Application Publication
(10) Pub. No.: US 2013/0296189 A1
(43) Pub. Date: Nov. 7, 2013

(54) PROBES UTILIZING UNIVERSAL TAGS, A KIT COMPRISING THE SAME AND DETECTION METHODS

(71) Applicant: SUZHOU INSTITUTE OF NANO-TECH AND NANO-BIONICS, CHINESE ACADEMY OF SCIENCES, Suzhou (CN)

(72) Inventors: Jiong LI, Suzhou (CN); Demin DUAN, Suzhou (CN); Kexiao ZHENG, Suzhou (CN); Rong CAO, Suzhou (CN); Li JIANG, Suzhou (CN); Zhuoxuan LV, Suzhou (CN); Fang BAO, Suzhou (CN); Weibing GU, Suzhou (CN)

(21) Appl. No.: 13/946,785

(22) Filed: Jul. 19, 2013

Related U.S. Application Data
(63) Continuation-in-part of application No. 13/255,881, filed on Nov. 14, 2011.

(30) Foreign Application Priority Data
May 8, 2009 (CN) 200910083561.1

(51) Int. Cl. C12Q 1/68

(52) U.S. Cl. CPC C12Q 1/6837
USPC 506/9; 506/16

(57) ABSTRACT

The present invention provides a kit and a detection method for multiple targets detection of biomolecules. The kit comprises a universal tag, a probe and an optional instruction for using the same. The universal tag in the present invention is a fragment of DNA, RNA, peptide nucleic acid, or LNA, and is 3-20 mer in length. The probe in the present invention contains in order from 3′ terminus to 5′ terminus, a nucleotide sequence which is reverse complementary to a target molecule or a portion of the target molecule, and a nucleotide sequence which is reverse complementary to the universal tag; or said probe contains in order from 3′ terminus to 5′ terminus, a nucleotide sequence which is reverse complementary to the universal tag, and a nucleotide sequence which is reverse complementary to a target molecule or a portion of the target molecule.

### CROSS REFERENCE TO RELATED APPLICATIONS

The present application is a continuation in part application of U.S. application Ser. No. 13/255,881, filed Nov. 14, 2011, which is a national phase application under 35 U.S.C. §371 of International Application No. PCT/CN2010/000541 filed Apr. 20, 2010, which claims priority to Chinese Application No. 200910083561.1, filed May 8, 2009. The contents of the referenced applications are incorporated by reference.

### FIELD OF THE INVENTION

The present invention relates to the field of detection technology for biomolecules. Particularly, the present invention relates to probes utilizing universal tags, a kit comprising the same, and detection methods for multiple targets detection of biomolecules.

### BACKGROUND OF THE INVENTION

With the completion of human genome project (HGP), a large number of genome sequences of animals, plants and microorganisms have been determined and the genomic data are increasing in an unprecedented speed. Since the number of genes and other regulatory elements (such as microRNAs) is enormous, how to study the genetic and epigenetic information in a large scale and to analyze their biological functions during the life process have become a hot subject for scientists and researchers in academic, industrial and medical sectors. Under the background described above, the development and widespread application of high-throughput biosensors based on gene chip techniques have been deemed as one of the most significant technological progresses since the middle 1990s [1-4]. Gene chips, also known as DNA chips, DNA microarrays, or oligonucleotide arrays, refer to an two-dimensional array of thousands to millions of DNA probe spots, which is generated by in situ synthesis or automated micro-spotting to fix thousands of individual DNA probe molecules of a pre-defined sequence on each submillimeter-sized spot on the surface of a solid phase support. The primary application of DNA microarrays is to exact information of molecular abundance in a given sample via the hybridization between immobilized probes and labeled nucleic acids in a biological or medical specimen, based on Watson-Crick base pairing principal. Due to the sheer number of different probe spots on the microarray, the detection of the biological specimen proceeds in a parallel and highly-efficient fasion, i.e. thousands or millions of target molecules could be assayed simutaneously by analyzing the hybridization signals. So far, gene chip techniques have been widely used in the molecular biology, the medical research and so on, and have shown a good prospect of application in the fields such as gene expression, single nucleotide polymorphism (SNP), genome research, disease diagnosis, and drug screening and so on.

Although gene chips have proved their superior advantages in high-throughput parallel detection of numerous target molecules, there are some bottleneck factors limiting the practical application and popularization of gene chips. An important factor is that the samples to be tested need to be labeled before hybridization, and the steps of labeling samples are tedious, expensive, and need to be operated by professionals. Many labeling methods demands the usage of protein enymes such as reverse transcriptases, polymerases, etc. The labeling efficiency is relatively low,and the process can not be performed on the detection site. The chemical-labeling approaches requires no participation of enzymes, however, they are prone to bias (some bases get higher labeling efficiency than others). All of these factors have increased the detection cost and the operation steps and are disadvantageous to the popularization and the practical application of chip techniques. The object of the present invention is to overcome one or more defects of current labeling methods, and to provide a convenient and cost-efficient detection method by employing a universal tag based, label-free strategy.

It is well known that there are two main factors for stabilizing nucleic acid double-helixes. One of the factors is the hydrogen bonds formed between the complementary base pairs, which mainly maintains the transversal stability of nucleic acid double-helixes. Another is the effect of the base stacking between the adjacent bases located on the same nucleic acid chain, which is the major factor for maintaining longitudinal stability of nucleic acid double-helixes. The two factors are functioning synergically to maintain the stability of nucleic acid double-helixes, wherein the forming of hydrogen bonds is helpful to the base stacking, while the base stacking is also helpful to the forming of hydrogen bonds. A research team led by Professor Mirzabekov of Russia's National Academy of Sciences, has systematically studied and explained the theory of base stacking hybridization (BSH) [5-9]. Base stacking hybridization is also named as contiguous stacking hybridization (CSH), and refers to that, when a short oligonucleotide single strand hybridizes with a complementary DNA/RNA long chain, the formed double-strand structure is usually unstable. However, if another oligonucleotide single strand adjacent to the short oligonucleotide single strand also hybridizes with the complementary DNA/RNA long chain, the stability of such double-strand structure will be greatly increased (FIG. 1). Based on the research results described above, the present invention provides a universal labeling method for multiple targets detection of biomolecules.

### DESCRIPTION OF THE INVENTION

An object of the present invention is to provide a universal tag for multiple targets detection of biomolecules.

Another object of the present invention is to provide a probe for multiple targets detection of biomolecules.

Another object of the present invention is to provide a multiple targets detection method of biomolecules.

Another object of the present invention is to provide a kit for multiple targets detection of biomolecules, wherein the kit comprises a universal tag, a probe and an optional instruction for using the kit.

The universal tag for multiple targets detection of biomolecules according to the present invention may be a fragment of DNA, RNA, PNA (peptide nucleic acids), LNA (Locked nucleic acids) and so on. The length of the universal tag varies in the range of 3-20 nucleiotides. On designing, the base sequence of the universal tag should be compared with that of the samples to be tested to minimize the sequence homology or similarity with the samples to be tested as much as possible.

The universal tags of the present invention can be labeled with indicators such as fluorescent dyes, quantum dots, nanogolds, isotopes, enzymes and biotins etc, so that they can be detected by means of fluorescence microscope, array scanner, silver staining coloration method, enzyme reaction coloration and electrochemical method etc. The method for labeling the universal tags with the indicators can be any conventional methods used in the art. For example, the indicators can be connected to the universal tags via a chemical reaction between function groups on the indicators and the termini (e.g., amine groups, carboxyl groups etc.) of the universal tags.

The indicators labeled to the universal tag can be easily detected by means of fluorescence microscope, array scanner, silver staining coloration method, enzyme reaction coloration method and electrochemical etc. The detecting methods per se are conventional methods in the art. Based on the specific indicators used, a person skilled in the art can easily select and use a suitable method to detect the signals produced by the indicators, so as to determine the concentration of the target molecules or the existance of the target molecules based on the intensity of the signals. For example, if the indicator is a fluorescent dye, a person skilled in the art can easily detect the existence of the fluorescence dye via the fluorescence microscope, and to further determine the concentration of the target molecules or the existance of the target molecules based on the intensity of the detected fluorescence signals.

The probe for multiple targets detection of biomolecules according to the present invention contains in order from 3′ terminus to 5′ terminus, a nucleotide sequence which is reverse complementary to the target molecule or a portion of the target molecule, and a nucleotide sequence which is reverse complementary to the universal tag, and a fragment of poly (T) or poly (A) optionally added on the 3′ terminus to reduce the interface influence of the solid phase support. Alternately, the probe contains in order from 3′ terminus to 5′ terminus, a nucleotide sequence which is reverse complementary to the universal tag, a nucleotide sequence which is reverse complementary to the target molecule or a portion of the target molecule, and a fragment of poly (T) or poly (A) optionally added on the 5′ terminus to reduce the interface influence of the solid phase support.

In another embodiment, the probe for multiple targets detection of biomolecules according to the present invention contains in order from 3′ terminus to 5′ terminus, a nucleotide sequence which is reverse complementary to the target molecule or a portion of the target molecule, and a nucleotide sequence which is reverse complementary to the universal tag, and a fragment of poly (T) or poly (A) optionally added on the 3′ terminus to reduce the interface influence of the solid phase support, and the probe is linked to the solid phase support at 3′ terminus optionally via the fragment of poly (T) or poly (A). Alternately, the probe contains in order from 3′ terminus to 5′ terminus, a nucleotide sequence which is reverse complementary to the universal tag, a nucleotide sequence which is reverse complementary to the target molecule or a portion of the target molecule, and a fragment of poly (T) or poly (A) optionally added on the 5′ terminus to reduce the interface influence of the solid phase support, and the probe is linked to the solid phase support at 5′ terminus optionally via the fragment of poly (T) or poly (A).

When the probe is linked to the solid phase support via the fragment of poly (T) or poly (A), the other portion of the probe can be spaced away from the solid phase support surface, i.e., the fragment of poly (T) or poly (A) acts as a spacer arm. Thus, interface influence of the solid phase support can be reduced.

In the probe according to the present invention, the terminal group is amine, thiol, carboxyl, or biotin etc. The probe of the present invention can be linked to the solid phase support via these terminal groups, e.g., via amine, thiol, carboxyl or biotin etc.

The probe of the present invention can be linked to the solid phase support via conventional methods in the art, e.g., via corresponding chemical reactions between the terminal group of the probe and the function group on the surface of the solid phase support.

The multiple targets detection method of biomolecules according to the present invention comprises following steps:

1) preparing the universal tags, wherein the universal tags may be labeled with indicators such as fluorescent dyes, quantum dots, nanogolds, isotopes, and biotins etc, so that they are suitable to be detected by means of fluorescence microscope, array scanner, silver staining coloration method, enzyme reaction coloration method etc;

2) preparing the probe as described above, wherein, firstly the probe is designed depending on the target to be detected, and the probe contains a fragment of nucleotide sequence which is reverse complementary to the above universal tags in addition to a fragment of nucleotide sequence which is reverse complementary to the target molecule or a portion of the target molecule. The terminus of the probe is modified in order to connect with the solid phase support, preferably via the fragment of poly (T) or poly (A) connected at the terminus of the probe;

3) linking the probe to a modified solid phase support;

4) dissolving the universal tags and the pre-treated sample to be tested into a hybridization solution, and hybridizing the universal tags and the sample with the probe array (specifically, applying the hybridization solution to the probe array, and allowing the molecular interactions (nucleic acid hybridization in particular) of immobilized probes, sample molecules and universal tags to proceed under controlled reaction conditions. Alternately, the process can be performed in two steps, i.e., first hybridizing the sample to be tested with the probe array, rinsing the sample, then hybridizing the universal tags with the probe array;

4) rinsing to remove the redundant sample and the redundant universal tags;

5) detecting and analyzing the hybridization signals, preferably detecting the signals produced by the indicators labeled on the universal tag with fluorescence microscope, a flat scanner, or an array scanner, and then determining the existence of the indicators labeled on the universal tag based on the detected signals. Thus, in the present invention, the existence of the target biomolecules can be easily determined based on the determination of the existence of the universal tag. And the concentration of the target molecules can be further determined based on the detected intensity of the signals.

In a preferred method, the above step 5) is performed with fluorescence microscope, an array scanner or a flat scanner.

In a further preferred method, the universal tags are labeled with a fluorescent dye, and the signal produced by the fluorescent dye are detected by fluorescence microscope or array scanner. The existence of the target molecules can be determined based on the detection of the fluorescent signals. That is to say, if fluorescent signals of the universal tag are detected, the existence of the target molecules can be determined. And the concentration of the target molecules can be further determined based on the detected intensity of the signals.

Without being bounded to the specific theory, the inventors hold that the present invention can determine the existence of the target molecules by the detection of the signals produced by the indicators labeled to the tag based on the mechanism as follows. In the present invention, when the universal tag is hybridized to the probe either simultaneously with or after the hybridization of the target molecules, an effect of base stacking hybridization will occur. Because of the effect of base stacking hybridization, the universal tag can bind to the probes steadily. Otherwise the universal tag can not bind to the probe steadily, because the universal tag is relatively short and is labeled with indicators, which prevents the universal tags from steadily binding to the probe. That is to say, the universal tag of the present invention can bind to the probes steadily only when the completely complementary target molecules bind to the probes. When the mismatched target molecules bind to the probes, the binding between the universal tags and the probes can not be stabilized. If the universal tags are not bound to the probes steadily, they will be washed away in the following rinsing step, and thus, in the above step 5), no signals produced by the universal tags can be detected.

In the method according to the present invention, the subject to be detected includes not only DNAs and RNAs, but also proteins, saccharide molecules, etc., that can bind to the fragment of nucleotide sequence of the probe which is reverse complementary to the target molecule or a portion of the target molecule.

In the method according to the present invention, the solid phase support can be any solid support provided that the solid phase support does not disturb the detecting of the present invention substantially, for example, the solid phase support can be glass slide, plastic substrate, silicon wafer, microbeads, or polymer membrane, etc.

In the method according to the present invention, the solid phase support may be modified with poly-L-lysine, aldehyde group, carboxyl, or thiol, etc., so as to facilitate the connection of the probe to the solid phase support.

When detection is performed by using the universal tags and probes of the present invention, the detection can be performed directly without labeling after a sample is obtained, which greatly reduces the cost and is beneficial for in situ detection. The experimental procedure is simplified and a nonprofessional can operate since it is easy to operate, so it is convenient for the popularization of the technology. Furthermore, multiple targets detection of biomolecules can be achieved by using the tags and the probes of the present invention.

### BRIEF DESCRIPTION OF THE DRAWINGS

FIG. 1 is a schematic diagram illustrating the base stacking hybridization.

FIG. 2 is a schematic diagram illustrating the universal labeling method used in the detection of various clinical pathogens.

FIG. 3 is a schematic diagram illustrating the universal labeling method used in microRNA expression profiling.

FIG. 4 is a schematic diagram illustrating the universal labeling method used in the multiple detection of protein targets.

FIG. 5 is a schematic diagram illustrating the universal labeling method used in microRNA expression profiling.

FIG. 6 is a schematic diagram showing the result of testing example 1.

FIG. 7 is a schematic diagram showing the result of testing example 2.

FIG. 8 is a schematic diagram showing the result of testing example 4.

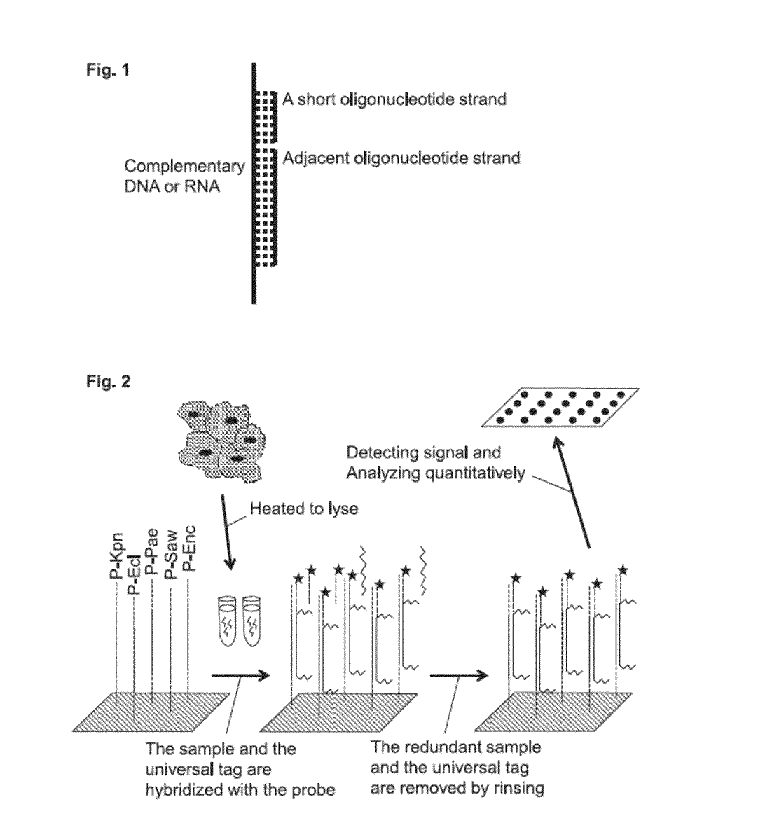

### DETAILED DESCRIPTION OF THE EMBODIMENTS

In the present invention, “hybridizing” means the action of mixing so as to facilitate the specific binding interactions between target molecules and corresponding probes or between the probes and the universal tags. The outcome of successful hybridization is the formation of thermodynamically stable molecular complexes.

In the present invention, “fluorescent dye” means a signaling organic molecule that can emit photons of a longer wavelength upon specific light excitation.

In the present invention, “flat scanner” means a commercial office scanner that is mainly used to acquire opitcal images from surfaces of various written or printed materials, such as paper books, documents, and photos, etc.

In the present invention, “array scanner” means a commercial microarray scanner equipped with laser light source that is used to acquire the fluorescent images from microarray slides.

In the present invention, “quantum dot” means a semiconductor nanocrystal whose excitons are confined in all spatial dimensions. Due to the quantum confinement, the quantum dots (or QDs) are able to develop intense and long-lasting fluorescence excitable by various light sources.

In the present invention, “nanogold” means a gold nanoparticle that has a diameter of less than 100 nanometers.

In the present invention, “micorbeads” means inorganic or polymer particles of uniform size and shape, typically 0.5 to 500 micrometers in diameter, whose surface could be decorated with specific molecular ligands (such as DNA or antibodies) via adsorption or chemical coupling for the purposes of isolation, purification or analyzing specific molecules.

The following examples are for illustrating the present invention, and can not be construed as limiting the scope of protection of the present invention.

### EXAMPLES

The following examples are used to illustrate the principle of the present invention, and can not be construed as limiting the scope of protection of the present invention.

### Example 1

### Application of the Method According to the Present Invention in the Detection of Various Clinical Pathogens

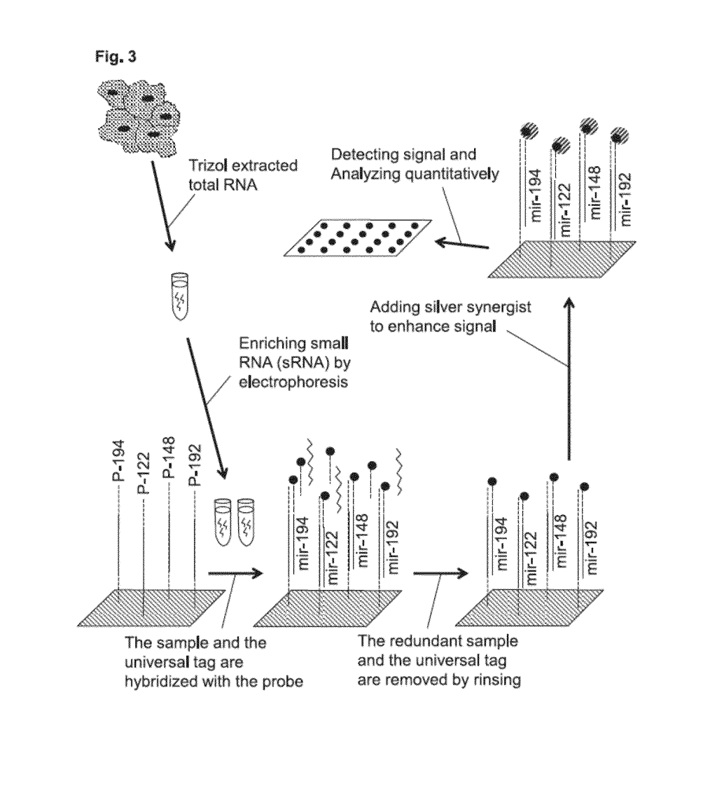

Five pathogens obtained from respiratory passage of pneumonia are taken as examples (shown in FIG. 2) to describe the Example 1: K. pneumoniae, E. cloacae, P. aeruginosa, S. aureus, and Enterococcus. The probes and universal tags to be used are shown in Table 1.

TABLE 1
Names and sequences of the probes and the universal tag
used in the detection of the five pathogen samples
obtained from respiratory passage of pneumonia

| | targets | Sequences (5′-3′) |
|---|---|---|
| Probes | | |
| P-Kpn | K.pneumoniae | NH2-T12-AACCGCTGGCAACAAAG-TACGACACT |
| | 16SRNA | (SEQ ID NO: 1) |
| P-Ecl | E.cloacae | NH2-T12-GTAGGTAAGGTTCTTCG-TACGACACT |
| | 16SRNA | (SEQ ID NO: 2) |
| P-Pae | P.aeruginosa | NH2-T12-GCGCCCGTTTCCGGAC-TACGACACT |
| | 16SRNA | (SEQ ID NO: 3) |
| P-Sau | S.aureus 16SRNA | NH2-T12-AGCAAGCTTCTCGTCCG-TACGACACT |
| | | (SEQ ID NO: 4) |
| P-Enc | Enterococcus | NH2-T12-GTTTCCAAGTGTTATCCC-TACGACACT |
| | 16SRNA | (SEQ ID NO: 5) |
| universal tag | | |
| UT-16S | | FAM-AGTGTCGTA (SEQ ID NO: 6) |

1. The 16S RNAs of the five pathogens are chosen as the targets of detection, and five probes are synthesized, respectively, wherein the 5′ terminus fragment of the probe is poly (T) 12, the middle fragment of the probe is complementary to a portion of the target molecule, and the fragment of sequence on the 3′ terminus is complementary to the universal tag, and the 5′ terminus of the probe is modified with an amine group;

2. The universal tag is synthesized and is modified with fluorescein on its 5′ terminus;

3. A glass slide is treated by using conventional chemical modification method to prepare an aldehyde substrate;

4. The probe is dissolved in a spotting buffer solution, and then the oligonucleotide arrays are prepared by spotting;

5. The secretion substance from respiratory passage of a patient is heated to lyse, or the bacterial culture suspension is heated to lyse after the secretion substance is bacterial-cultured. Then the lysed substance is dissolved in hybridization solution together with the universal tag and hybridized with the probe arrays;

6. The redundant samples and the redundant universal tag are removed by rinsing;

7. The detection is performed by using fluorescence microscope or array scanner and analysis is performed. The detection is performed by conventional method in the art. Specifically, after hybridization, the fluorescent dye labeled universal tags is bound to microarrays. Then the microarrays are scanned to capture fluorescent images and then the fluorescence intensity is used to determine the concentrations of the targets.

Because of the effect of base stacking hybridization, the universal tag can be linked to the probes steadily only when the completely complementary target molecules are linked to the probes. When the mismatched target molecules are linked to the probes, the linkage between the universal tag and the probes can not be stabilized. The five probes are hybridized with the 16SRNAs of the five pathogens, respectively. Thus the types and contents of the infected pathogens can be determined to guide clinical medication.

### Example 2

### Application of the Method According to the Present Invention in the Analysis of miRNA Profile

Four miRNAs obtained from tissue of liver are taken as examples (shown in FIG. 3) to describe the Example 2: hsa-mir-194, hsa-mir-122, hsa-mir-148, and hsa-mir-192. The probes and universal tags to be used are shown in Table 2.

TABLE 2
Names and sequences of the probes and the universal tag
to be used in the detection of the four miRNAs obtained
from the tissue of liver

| | Targets | sequences (5′-3′) |
|---|---|---|
| Probes | | |
| P-194 | hsa-mir-194 | NH2-A10-TCCACATGGAGTTGCTGTTACA-TGCGACCTG |
| | | (SEQ ID NO: 7) |
| P-122 | hsa-mir-122 | NH2-A10-CAAACACCATTGTCACACTCCA-TGCGACCTG |
| | | (SEQ ID NO: 8) |
| P-148 | hsa-mir-148 | NH2-A10-ACAAAGTTCTGTAGTGCACTGA-TGCGACCTG |
| | | (SEQ ID NO: 9) |
| P-192 | hsa-mir-192 | NH2-A10-GGCTGTCAATTCATAGGTCAG-TGCGACCTG |
| | | (SEQ ID NO: 10) |
| universal tag | | |
| UT-miRNA | | nanogold-CAGGTCGCA (SEQ ID NO: 11) |

1. The probes corresponding to above four miRNAs are prepared according to miRNA library, wherein the 5′ terminus of the probe is poly (A) 10, a fragment of sequence in middle of the probe is complementary to the miRNA, a fragment of sequence on the 3′ terminus is complementary to the universal tag, and the 5′ terminus of the probe is modified with an amine group;

2. The universal tag is synthesized and is modified with nanogold on its 5′ terminus;

3. A glass slide is treated by using conventional chemical modification method to prepare an aldehyde substrate;

4. The probes are dissolved in a spotting buffer solution, and then the oligonucleotide arrays are prepared by spotting;

5. After the samples are lysed or total RNAs are extracted and small RNAs (sRNAs) are separated and enriched, the samples, together with the universal tag, are dissolved in a hybridization solution and hybridized with the probe;

6. The redundant samples and the redundant universal tag are removed by rinsing;

7. A silver synergist is added to enhance the signal;

8. The signals are detected and analyzed by using a flat scanner to determine the expression profile of the miRNA. The detection and analysis are performed by conventional method in the art. Specifically, the nanogold nucleates the highly specific deposition of silver from a silver synergist to form dark images, which can be scanned with a commercial office scanner. The intensities can be analyzed with commercial software to indicate the concentrations of the targets.

### Example 3

### Application of the Method According to the Present Invention in Multiple Detection for Protein Targets

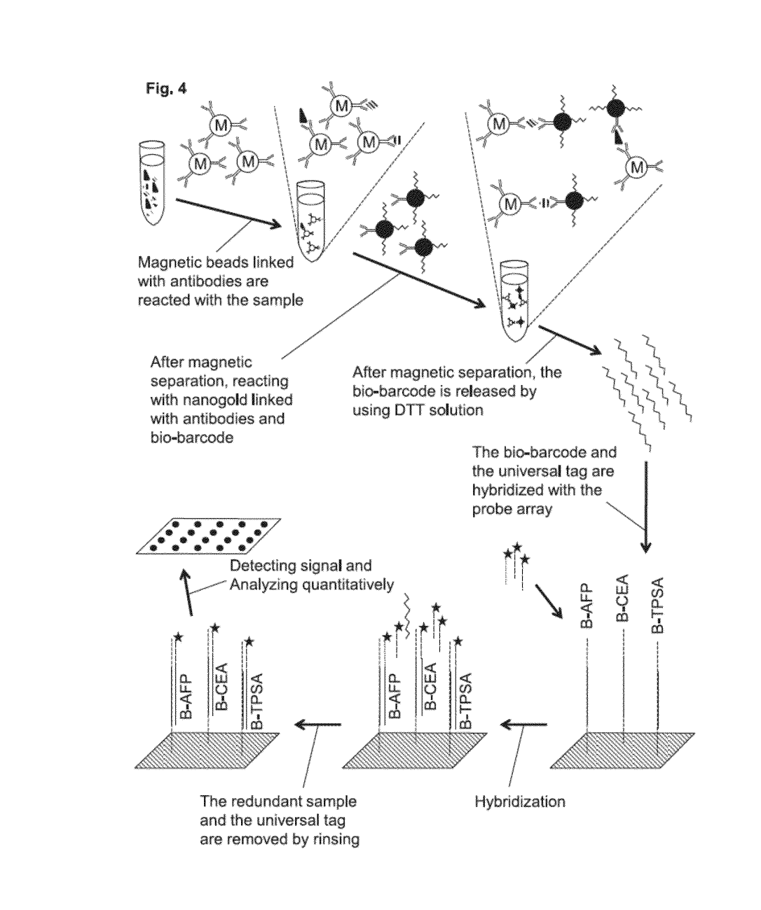

Alpha fetoprotein (AFP), carcino-embryonic antigen (CEA), and total prostate specific antigen (TPSA) which are obtained from human serum are taken as examples (shown in FIG. 4) to describe the Example 3. The probes, bio-barcodes, and universal tags to be used are shown in Table 3.

TABLE 3
Names and sequences of the probes, bio-barcodes, and universal tag
to be used in the detection of the three antigens from human serum

| | targets | sequences (5′-3′) |
|---|---|---|
| Probes | | |
| P-AFP | alpha fetoprotein | NH2-T10-CAGCATCGGACCGGTAATCG- |
| | | TACGACACT |
| | | (SEQ ID NO: 12) |
| P-CEA | carcino-embryonic | NH2-T10-TGCGATCGCAGCGGTAACCT- |
| | antigen | TACGACACT |
| | | (SEQ ID NO: 13) |
| P-TPSA | total prostate | NH2-T10-GACCATAGTGCGGGTAGGTA- |
| | specific antigen | TACGACACT |
| | | (SEQ ID NO: 14) |
| bio-bar code | | |
| B-AFP | alpha fetoprotein | CGATTACCGGTCCGATGCTG |
| | | (SEQ ID NO: 15) |
| B-CEA | carcino-embryonic | AGGTTACCGCTGCGATCGCA |
| | antigen | (SEQ ID NO: 16) |
| B-TPSA | total prostate | TACCTACCCGCACTATGGTC |
| | specific antigen | (SEQ ID NO: 17) |
| universal tag | | |
| UT-pro | | FAM-AGTGTCGTA |
| | | (SEQ ID NO: 18) |

1. The antibodies corresponding to the three antigens to be detected are linked to magnetic beads and the magnetic beads linked with antibodies are then reacted with sample solutions, so as to form the antigen-antibody complexes;

2. The redundant samples are removed by magnetic separation, and then the nanogolds modified with the antibody and bio-barcode (the three antigens to be detected are corresponded to three different barcode nucleotide sequences), are reacted with the antigen-antibody complexes, to form the complexes of magnetic bead-antigen-nanogold;

3. The redundant nanogold is removed by magnetic separation, and then the bio-barcodes are released from nanogold by using DTT solution;

4. The released bio-barcodes and the universal tags labeled with FAM are dissolved in hybridization solution and hybridized with the probe array (the 3′ terminus of the probe is complementary to the universal tag, the portion in middle of the probe is complementary to corresponding bio-barcode, the 5′ terminus is poly (T) 10, and the 5′ terminus is modified with an amine group so as to be fixed on the aldehyde glass slide);

5. The redundant universal tag is removed by rinsing;

6. The detection and analysis are performed by using a fluorescence microscope or an array scanner to determine the types and contents of the three antigens in the serum sample. Specifically, the detection and analysis are performed by conventional methods in the art, i.e., after hybridization, the fluorescent dye labeled universal tags are bound to microarrays, then the microarrays are scanned to capture fluorescent images and then the fluorescence intensity is used to determine the concentrations of the targets.

### Example 4

### Application of the Method According to the Present Invention in the Analysis of miRNA, which has High Specificity

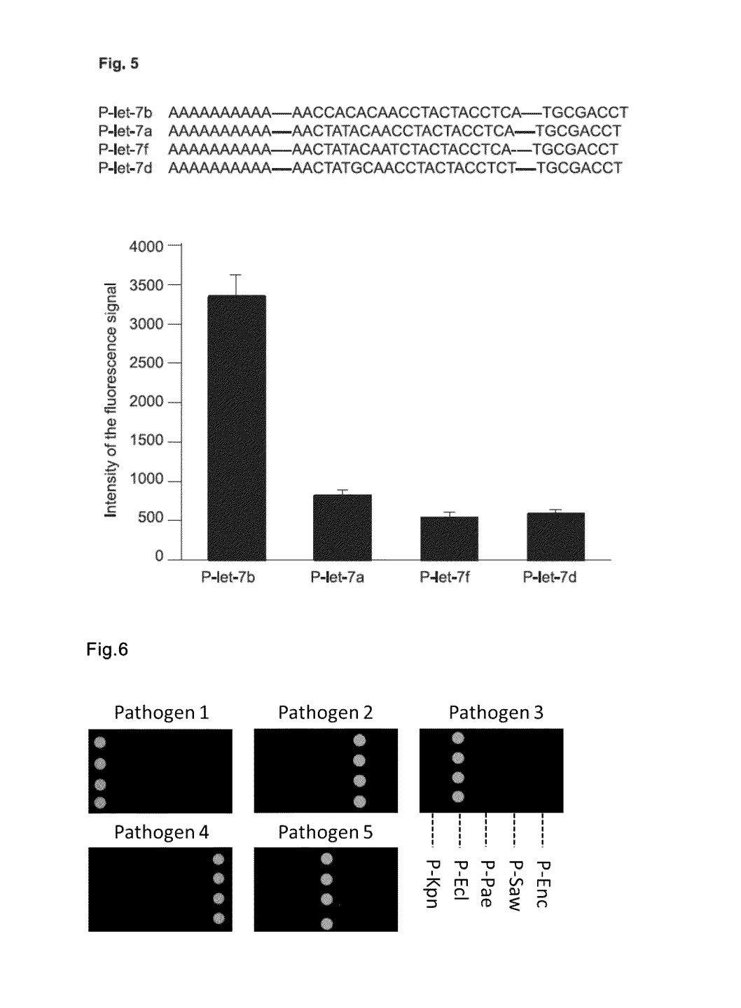

Four members hsa-let-7b, hsa-let-7a, hsa-let-7f, and hsa-let-7d of hsa-let-7 family of miRNA (shown in FIG. 5) are taken as examples to describe the Example 4, wherein the probes, universal tags, and targets to be used are shown in Table 4.

TABLE 4
Names and sequences of the probes, universal tags, and
targets used in the detection of the hsa-let-7 family

| Names | sequences (5′-3′) |
|---|---|
| probeP-let-7b | AAAAAAAAAA-AACCACACAACCTACTACCTCA-TGCGACCT |
| | (SEQ ID NO: 19) |
| probeP-let-7a | AAAAAAAAAA-AACTATACAACCTACTACCTCA-TGCGACCT |
| | (SEQ ID NO: 20) |
| probeP-let-7f | AAAAAAAAAA-AACTATACAATCTACTACCTCA-TGCGACCT |
| | (SEQ ID NO: 21) |
| probeP-let-7d | AAAAAAAAAA-AACTATGCAACCTACTACCTCT-TGCGACCT |
| | (SEQ ID NO: 22) |
| universal tag | AGGTCGCA (SEQ ID NO: 23) |
| target T-let-7b | Ugagguaguagguugugugguu |
| | (SEQ ID NO: 24) |

Note:
The bases represented with black body in the table are bases mismatched with the target T-let-7b.

1. Four probes corresponding to hsa-let-7b, hsa-let-7a, hsa-let-7f, and hsa-let-7d, respectively, are synthesized according to miRNA library, wherein the 5′ terminus of the probe is poly (A) 10, a fragment of sequence in the middle of the probe is complementary to the relevant miRNA, a fragment of sequence on the 3′ terminus is complementary to the universal tag, and the 5′ terminus of the probe is modified with amine groups;

2. The universal tag is synthesized, and the 5′ terminus is modified with luciferin Cy3;

3. The target T-let-7b is synthesized;

4. An aldehyde glass slide is prepared by using chemical modification method;

5. The probes are dissolved in a spotting buffer solution, and then, oligonucleotide arrays of four probes are prepared by spotting process;

6. Target T-let-7b and universal tag are dissolved in a hybridization solution and hybridized with the arrays;

7. The glass slide of arrays is rinsed;

8. The glass slide is scanned with scanner; (Specifically, after hybridization, the universal tags labeled with fluorescent dye are bound to microarrays. Then the microarrays are scanned to capture fluorescent images and the fluorescence intensity is used to determine the concentrations of the targets)

9. The result shows that the method of the invention has high specificity, and is able to identify targets which have only 2, 3, or 4 mismatched bases.

### TESTING EXAMPLES

The following testing examples are used to exemplify the present invention, and are not intended to limit the scope of the present invention in any sense.

### Example 5

### Application of the Method According to the Present Invention in the Detection of Various Clinical Pathogens

As used in above example 1, five pathogens obtained from respiratory passage of pneumonia are taken as examples (shown in FIG. 2) to describe Example 5: K. pneumoniae, E. cloacae, P. aeruginosa, S. aureus, and Enterococcus. The probes and universal tags to be used are shown in Table 5.

TABLE 5
Names and sequences of the probes and the universal tag used
in the detection of the five pathogen samples obtained from
respiratory passage of pneumonia

| | targets | Sequences (5′-3′) |
|---|---|---|
| Probes | | |
| P-Kpn | K. pneumoniae | NH2-T12-AACCGCTGGCAACAAAG-TACGACACT |
| | 16SRNA | (SEQ ID NO: 1) |
| P-Ecl | E.cloacae 16SRNA | NH2-T12-GTAGGTAAGGTTCTTCG-TACGACACT |
| | | (SEQ ID NO: 2) |
| P-Pae | P.aeruginosa | NH2-T12-GCGCCCGTTTCCGGAC-TACGACACT |
| | 16SRNA | (SEQ ID NO: 3) |
| P-Sau | S.aureus 16SRNA | NH2-T12-AGCAAGCTTCTCGTCCG-TACGACACT |
| | | (SEQ ID NO: 4) |
| P-Enc | Enterococcus | NH2-T12-GTTTCCAAGTGTTATCCC-TACGACACT |
| | 16SRNA | (SEQ ID NO: 5) |
| universal tag | | |
| UT-16S | | FAM-AGTGTCGTA |
| | | (SEQ ID NO: 6) |

1. 16S RNAs of the five pathogens are chosen as the targets of detection, and five probes are synthesized, respectively, wherein the 5′ terminus of the probe is poly (T) 12, a fragment of sequence in middle of the probe is complementary to a portion of the target molecule, a fragment of sequence on the 3′ terminus is complementary to the universal tag, and the 5′ terminus of the probe is modified with an amine group;

2. The universal tag is synthesized and is modified with fluorescein on its 5′ terminus;

3. A glass slide is treated by using conventional chemical modification method to prepare an aldehyde substrate;

4. The probe is dissolved in a spotting buffer solution (pH 9.0, 0.1 M sodium carbonate, 1.5 M betaine, 20% dimethyl sulfoxide) to prepare 20 μM working solution, and then the oligonucleotide arrays are printed by a commercial arrayer at 24-26° C. along with 50-55% relative humidity, and each probe was spotted in quadruplicate within an array;

5. The spotted slide is incubated in a humid chamber overnight at room temperature, then washed twice for 10 min in 0.1% sodium dodecyl sulfate (SDS), followed by thorough washing with ultrapure water;

6. The secretion substance from respiratory passage of a patient is heated to lyse, or the bacterial culture suspension is heated to lyse after the secretion substance is bacterial-cultured, and then cooled on ice immediately before assay. The lysed substance contained 0.1-2 μg DNA is dissolved in hybridization solution (5×saline-sodium citrate buffer (SSC) with 0.2% SDS) together with the 200 nM universal tag and hybridized with the probe arrays at 42° C. for 16 h in a hybridization oven with a constant rotation speed of 15 rpm;

7. After hybridization, slide is washed in 5×SSC and 0.1% SDS at 30° C. for 6 min, and then washed for 3 min twice at room temperature in 0.2×SSC. The slide is immediately spin-dried on a slide centrifuge. The redundant samples and the redundant universal tag are removed by rinsing;

8. The detection is performed by conventional method in the art: first imaging the slide with a fluorescence microscope or an microarray scanner, and then analyzing the acquired data. Specifically, after hybridization, the fluorescent dye-labeled universal tags is bound to microarray probes which specifically capture their molecular targets. Therefore, the fluorescence intensity of each probe spot (called “feature”) is quantitatively correlated to the concentration of its corresponding target in the sample used to determine the concentrations of the targets.

9. The raw fluorescence intensity is extracted using the GenePix Pro software, FAM median fluorescence intensity values are background subtracted. Statistical analysis is performed using OriginPro software and R statistical computing framework.

10. Because of the effect of base stacking hybridization, the universal tag can be linked to the probes steadily only when the completely complementary target molecules are linked to the probes. When the mismatched target molecules are linked to the probes, the linkage between the universal tag and the probes can not be stabilized. The five probes are hybridized with the 16SRNAs of the five pathogens, respectively. Thus the types of the infected pathogens can be determined to guide clinical medication. The experimental results (shown in FIG. 6) show the lysed substance of pathogen 1 hybridized with probe P-Kpn generates high signal, while other probes P-Ecl, P-Pae, P-Saw, and P-Enc are negligible. Similarly, the pathogen 2-5 generate high signal when they hybridized with probe P-Saw, P-Ecl, P-Enc, and P-Pae, respectively. It indicates the type of the pathogen 1-5 is K. pneumoniae, S. aureus, E. cloacae, Enterococcus, and P. aeruginosa, respectively.

### Example 6

### Application of the Method According to the Present Invention in the Analysis of miRNA Profile

As used in above example 2, four miRNAs obtained from tissue of liver are taken as examples (shown in FIG. 3) to describe Example 6: hsa-mir-194, hsa-mir-122, hsa-mir-148, and hsa-mir-192. The probes and universal tags to be used are shown in Table 6.

TABLE 6
Names and sequences of the probes and the universal tag
to be used in the detection of the four miRNAs obtained
from the tissue of liver

| | Targets | sequences (5′-3′) |
|---|---|---|
| Probes | | |
| P-194 | hsa-mir-194 | NH2-A10-TCCACATGGAGTTGCTGTTACA-TGCGACCTG |
| | | (SEQ ID NO: 7) |
| P-122 | hsa-mir-122 | NH2-A10-CAAACACCATTGTCACACTCCA-TGCGACCTG |
| | | (SEQ ID NO: 8) |
| P-148 | hsa-mir-148 | NH2-A10-ACAAAGTTCTGTAGTGCACTGA-TGCGACCTG |
| | | (SEQ ID NO: 9) |
| P-192 | hsa-mir-192 | NH2-A10-GGCTGTCAATTCATAGGTCAG-TGCGACCTG |
| | | (SEQ ID NO: 10) |
| universal tag | | |
| UT-miRNA | | nanogold-CAGGTCGCA (SEQ ID NO: 11) |

1. The probes corresponding to above four miRNAs are prepared according to miRNA library, wherein the 5′ terminus of the probe is poly (A) 10, a fragment of sequence in middle of the probe is complementary to the miRNA, a fragment of sequence on the 3′ terminus is complementary to the universal tag, and the 5′ terminus of the probe is modified with an amine group;

2. The universal tag is synthesized and is modified with nanogold on its 5′ terminus;

3. A glass slide is treated by using conventional chemical modification method to prepare an aldehyde substrate;

4. The probes are dissolved in a spotting buffer solution (pH 9.0, 0.1 M sodium carbonate, 1.5 M betaine, 20% dimethyl sulfoxide) to prepare 20 μM working solution, and then the oligonucleotide arrays are printed by a commercial arrayer at 24-26° C. along with 50-55% humidity, each probe is spotted in quadruplicate within an array;

5. The spotted slide is incubated in a humid chamber overnight at room temperature, then washed twice for 10 min in 0.1% sodium dodecyl sulfate, followed by thorough washing with ultrapure water;

6. After the samples are lysed or total RNAs are extracted and small RNAs (sRNAs) are separated and enriched, the samples containing 0.1-2 μg RNA, together with the 200 nM universal tag, are dissolved in a hybridization solution (5×SSC and 0.2% SDS) and hybridized with the probe at 42° C. for 16 h in a hybridization oven with a constant rotation speed of 15 rpm;

7. After hybridization, slide is washed in 5×SSC and 0.1% SDS at 30° C. for 6 min, and then washed for 3 min, twice at room temperature in 0.2×SSC. The slide is immediately dried on a slide centrifuge. The redundant samples and the redundant universal tag are removed by rinsing;

8. A silver synergist is added to enhance the signal using the silver enhancer kit of Sigma. Add enough silver enhancer mixture (˜2 ml) to cover the microarray surface. Develop at 20° C. for 5-10 minutes until the desired stain intensity is reached. Quench the reaction when the desired stain intensity is reached by rinsing the slide in distilled water for 3 min twice at room temperature. Then the slide is immediately dried on a slide centrifuge;

9. The signals are detected and analyzed by using a flat scanner to determine the expression profile of the miRNA. The detection and analysis are performed by conventional method in the art.

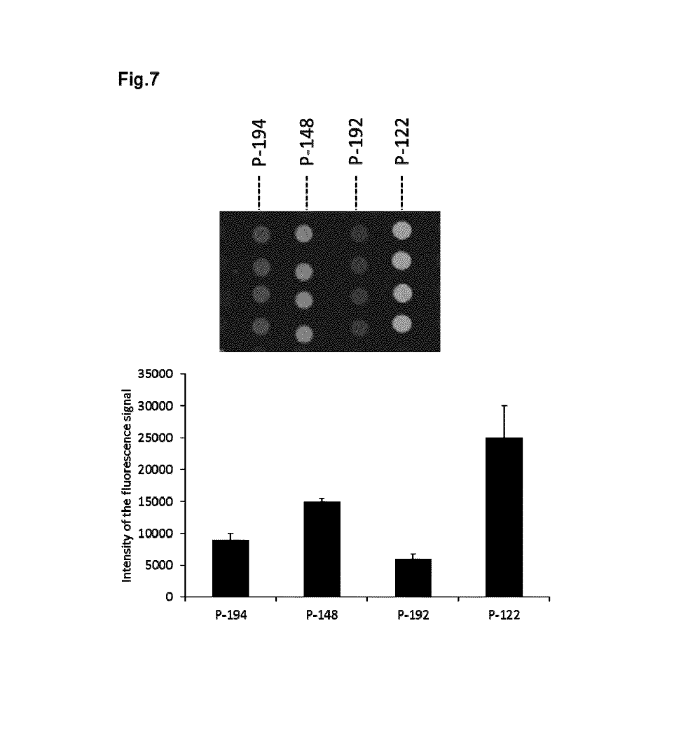

10. Specifically, the nanogold nucleates the highly specific deposition of silver from a silver synergist to form dark images, which can be scanned with a commercial office scanner. The intensities can be analyzed with commercial software to acquire the concentrations of the targets. The experimental results (shown in FIG. 7) show the expression profileg of these four miRNAs in liver tissue are different, and the concentration of the targets is miR-122>miR-148>miR-194>miR-192.

### Example 7

### Application of the Method According to the Present Invention in Multiple Detection for Protein Targets

As in above example 3, alpha fetoprotein (AFP), carcino-embryonic antigen (CEA), and total prostate specific antigen (TPSA) which are obtained from human serum are taken as examples (shown in FIG. 4) to describe the Example 7. The probes, bio-barcodes, and universal tags to be used are shown in Table 7.

TABLE 7
Names and sequences of the probes, bio-barcodes, and
universal tag to be used in the detection of the three
antigens from human serum

| | targets | sequences (5′-3′) |
|---|---|---|
| Probes | | |
| P-AFP | alpha fetoprotein | NH2-T10-CAGCATCGGACCGGTAATCG- |
| | | TACGACACT |
| | | (SEQ ID NO: 12) |
| P-CEA | carcino-embryonic | NH2-T10-TGCGATCGCAGCGGTAACCT-TACGACACT |
| | antigen | (SEQ ID NO: 13) |
| P-TPSA | total prostate | NH2-T10-GACCATAGTGCGGGTAGGTA- |
| | specific antigen | TACGACACT |
| | | (SEQ ID NO: 14) |
| bio-bar code | | |
| B-AFP | alpha fetoprotein | CGATTACCGGTCCGATGCTG |
| | | (SEQ ID NO: 15) |
| B-CEA | carcino-embryonic | AGGTTACCGCTGCGATCGCA |
| | antigen | (SEQ ID NO: 16) |
| B-TPSA | total prostate | TACCTACCCGCACTATGGTC |
| | specific antigen | (SEQ ID NO: 17) |
| universal tag | | |
| UT-pro | | FAM -AGTGTCGTA (SEQ ID NO: 18) |

1. The antibodies corresponding to the three antigens to be detected are linked to magnetic beads. The commercial streptavidin-modified magnetic beads are mixed with 50 μl three different biotin-modified antibodies (the concentration of AFP, CEA, and TPSA is 10 μg/ml, 5 μg/ml, and 2.5 μg/ml, respectively) for 4 h, respectively, then wash and suspend in PBS buffer. The magnetic beads linked with antibodies are then reacted with 2-50 μg/ml sample solutions for 30 min, so as to form the antigen-antibody complexes. The redundant samples are removed by magnetic separation;

2. About 5 ml nanogolds (pH 9.0) mix well with 1-10 μg/ml antibody and 10-20 μg/ml bio-barcode for 3 h and then react overnight at 4° C. (the three antigens to be detected are corresponded to three different barcode nucleotide sequences). The redundant antibody and the redundant bio-barcode are removed by rinsing at 12000 rpm/min. The modified nanogolds are reacted with the antigen-antibody complexes (the modified magnetic bead mentioned above) for 20 min in PBS solution to form the complexes of magnetic bead-antigen-nanogold;

3. The redundant nanogold is removed by magnetic separation, and then the bio-barcodes are released from nanogold by using 100 μl 1M DTT solution for 3 h at room temperature;

4. A glass slide is treated by using conventional chemical modification method to prepare an aldehyde substrate. The probes are dissolved in a spotting buffer solution (pH 9.0, 0.1 M sodium carbonate, 1.5 M betaine, 20% dimethyl sulfoxide) to prepare 20 μM working solution, and then the oligonucleotide arrays are printed by a commercial arrayer prepared by spotting at 24-26° C. along with 50-55% humidity, each probe was spotted in quadruplicate within an array;

5. The spotted slide is incubated in a humid chamber overnight at room temperature, then wash twice for 10 min in 0.1% sodium dodecyl sulfate, followed by thorough washing with ultrapure water;

6. The released bio-barcodes and 200 nM universal tags labeled with FAM are dissolved in hybridization solution (5×SSC and 0.2% SDS) and hybridized with the probe array (the 3′ terminus of the probe is complementary to the universal tag, the portion in middle of the probe is complementary to corresponding bio-barcode, the 5′ terminus is poly (T) 10, and the 5′ terminus is modified with an amine group so as to be fixed on the aldehyde glass slide) at 42° C. for 16 h in a hybridization oven with a constant rotation speed of 15 rpm;

7. After hybridization, slide is washed in 5×SSC and 0.1% SDS at 30° C. for 3 min, and then washed for 1 min twice at room temperature in 0.2×SSC. The slide is immediately dried on a slide centrifuge. The redundant universal tag is removed by rinsing;

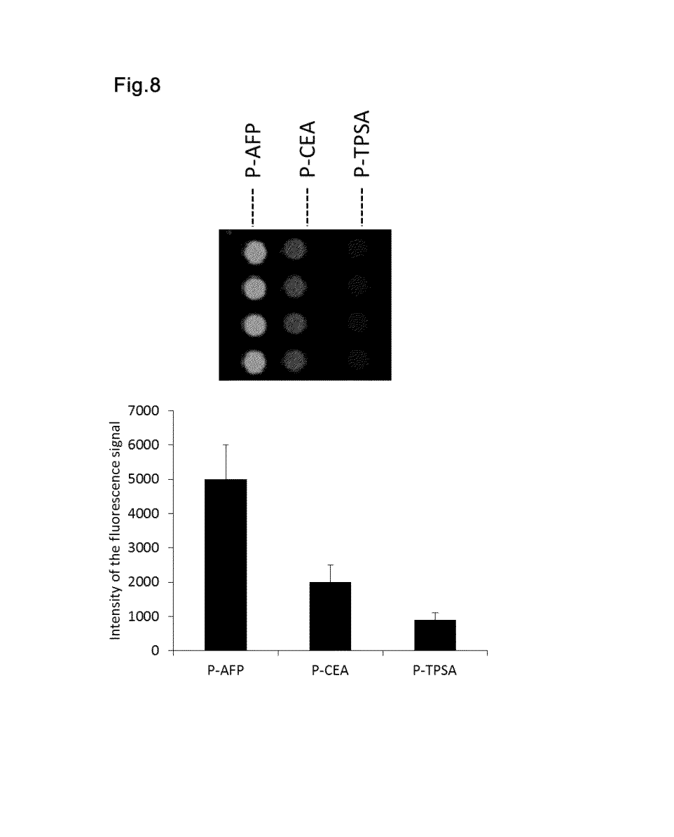

8. The detection and analysis are performed by using a fluorescence microscope or an array scanner to determine the types and contents of the three antigens in the serum sample. Specifically, the detection and analysis are performed by conventional methods in the art. That is to say, after hybridization, the fluorescent dye labeled universal tags are bound to microarrays, then the microarrays are scanned to capture fluorescent images and then the fluorescence intensity is used to determine the concentrations of the targets. The experimental results (shown in FIG. 8) show the fluorescence intensity of P-AFP, P-CEA, and P-TPSA are ˜5000, ˜2000, and ˜1000, respectively. Generally, it reflects the concentration of the targets (AFP, CEA, and TPSA are 10 μg/ml, 5 μg/ml, and 2.5 μg/ml, respectively).

### Example 8

### Application of the Method According to the Present Invention in the Analysis of miRNA, which has High Specificity

As in above example 4, four members hsa-let-7b, hsa-let-7a, hsa-let-7f, and hsa-let-7d of hsa-let-7 family of miRNA (shown in FIG. 5) are taken as examples to describe Example 8, wherein the probes, universal tags, and targets to be used are shown in Table 8.

TABLE 8
Names and sequences of the probes, universal tags, and
targets used in the detection of the hsa-let-7 family

| Names | sequences (5′-3′) |
|---|---|
| probeP-let-7b | AAAAAAAAAA-AACCACACAACCTACTACCTCA-TGCGACCT |
| | (SEQ ID NO: 19) |
| probeP-let-7a | AAAAAAAAAA-AACTATACAACCTACTACCTCA-TGCGACCT |
| | (SEQ ID NO: 20) |
| probeP-let-7f | AAAAAAAAAA-AACTATACAATCTACTACCTCA-TGCGACCT |
| | (SEQ ID NO: 21) |
| probeP-let-7d | AAAAAAAAAA-AACTATGCAACCTACTACCTCT-TGCGACCT |
| | (SEQ ID NO: 22) |
| universal tag | AGGTCGCA (SEQ ID NO: 23) |
| target T-let-7b | Ugagguaguagguugugugguu (SEQ ID NO: 24) |

Note:
The bases represented with black body in the table are bases mismatched with the target T-let-7b.

1. Four probes corresponding to hsa-let-7b, hsa-let-7a, hsa-let-7f, and hsa-let-7d, respectively, are synthesized according to miRNA library, wherein the 5′ terminus of the probe is poly (A) 10, a fragment of sequence in the middle of the probe is complementary to the relevant miRNA, a fragment of sequence on the 3′ terminus is complementary to the universal tag, and the 5′ terminus of the probe is modified with amine groups;

2. The universal tag is synthesized, and the 5′ terminus is modified with luciferin Cy3;

3. The target RNA T-let-7b is synthesized from TaKaRa Biotechnology;

4. An aldehyde glass slide is prepared by using conventional chemical modification method;

5. The probes are dissolved in a spotting buffer solution (pH 9.0, 0.1 M sodium carbonate, 1.5 M betaine, 20% dimethyl sulfoxide) to prepare 20 μM working solution, and then, oligonucleotide arrays of four probes are printed by a commercial arrayer, at 24-26° C. along with 50-55% humidity, each probe was spotted in quadruplicate within an array;

6. The spotted slide is incubated in a humid chamber overnight at room temperature, then wash twice for 10 min in 0.1% sodium dodecyl sulfate, followed by thorough washing with ultrapure water;

7. Target T-let-7b (20 pM) and universal tag (200 nM) are dissolved in a hybridization solution (5×SSC and 0.2% SDS) and hybridized with the arrays at 42° C. for 16 h in a hybridization oven with a constant rotation speed of 15 rpm;

8. After hybridization, slide is washed in 5×SSC and 0.1% SDS at 30° C. for 6 min, and then wash for 3 min twice at room temperature in 0.2×SSC. The slide is immediately dried on a slide centrifuge. The glass slide of arrays is rinsed;

9. The glass slide is scanned with the microarray scanner at constant power and PMT gain settings through a single-color channel (532nm wavelength) to capture fluorescent images; (Specifically, after hybridization, the universal tags labeled with fluorescent dye are bound to microarrays. Then the microarrays are scanned to capture fluorescent images and the fluorescence intensity is used to determine the concentrations of the targets);

10. The raw fluorescence intensity is extracted using the GenePix Pro software, FAM median fluorescence intensity values are background subtracted. Statistical analysis is performed using OriginPro software.

The result shows that the method of the invention has high specificity, the cross-hybridizations (target is let-7b, and four probes are P-let-7b, P-let-7a, P-let-7f, and P-let-7d) observed are under 30%. It indicates the method is able to distinguish targets which have only 2, 3, or 4 mismatched bases (the difference between let-7b and let-7a, let-7f, let-7d is 2, 3, 4 bases mismatch, respectively).

### REFERENCES

[1] Fodor S P, Read J L, Pirrung M C, Stryer L, Lu A T, Solas D. Light-directed, spatially addressable parallel chemical synthesis. Science 1991 Feb. 15; 251(4995): 767-773.

[2] Breakthrough of the year. The runners-up. Science. 1998 Dec. 18; 282(5397): 2157-2161.

[3] Marshall A, Hodgson J. DNA chips: an array of possibilities. Nat Biotechnol. 1998 January; 16(1): 27-31.

[4] Service R F. Microchip arrays put DNA on the spot. Science. 1998 Oct. 16; 282(5388): 396-399.

[5] Yershov G, Barsky V, Belgovskiy A, Kirillov E, Kreindlin E, Ivanov I, Parinov S, Guschin D, Drobishev A, Dubiley S, Mirzabekov A. DNA analysis and diagnostics on oligonucleotide microchips. Proc Natl Acad Sci U S A. 1996 May 14; 93(10):4913-4918.

[6] Parinov S, Barsky V, Yershov G, Kirillov E, Timofeev E, Belgovskiy A, Mirzabekov A. DNA sequencing by hybridization to microchip octa-and decanucleotides extended by stacked pentanucleotides. Nucleic Acids Res. 1996 Aug. 1; 24(15):2998-3004.

[7] Dubiley S, Kirillov E, Lysov Y, Mirzabekov A. Fractionation, phosphorylation and ligation on oligonucleotide microchips to enhance sequencing by hybridization. Nucleic Acids Res. 1997 Jun. 15; 25(12):2259-2265.

[8] Parallel thermodynamic analysis of duplexes on oligodeoxyribonucleotide microchips. Fotin A V, Drobyshev A L, Proudnikov D Y, Perov A N, Mirzabekov A D. Nucleic Acids Res. 1998 Mar. 15; 26(6):1515-1521.

[9] Vasiliskov V A, Prokopenko D V, Mirzabekov A D. Parallel multiplex thermodynamic analysis of coaxial base stacking in DNA duplexes by oligodeoxyribonucleotide microchips. Nucleic Acids Res. 2001 Jun. 1; 29(11): 2303-2313.

1. A multiple targets detection method of biomolecules,
characterized in that the method comprises steps of:
1) preparing universal tags labeled with indicators, wherein the indicators are selected from the group consisting of fluorescent dyes, quantum dots, nanogolds, isotopes, biotins, and combinations thereof;
2) preparing probes;
3) linking the probes to a modified solid phase support to form probe arrays;
4) dissolving the universal tags and samples to be tested into a hybridization solution to hybridize with the probe arrays; or hybridizing the samples to be tested with the probes first, then hybridizing the universal tags with the probe arrays after rinsing;
5) rinsing to remove the redundant samples and the redundant universal tags; and
6) detecting the presence or absence of the indicators on the probe arrays with a fluorescence microscope, a flat scanner, or an array scanner, wherein:
the biomolecules are selected from the group of molecules consisting of DNA, RNA, protein and/or saccharide;
the universal tag is a fragment of DNA, RNA, peptide nucleic acid, or LNA, and is 3-20 mer in length;
the probe contains in order from its 3′ terminus to 5′ terminus, a nucleotide sequence which is reverse complementary to a target molecule or a portion of a target molecule, and a nucleotide sequence which is reverse complementary to the universal tag, and a fragment of poly (T) or poly (A) optionally added on the 3′ terminus; or the probe contains in order from its 3′ terminus to 5′ terminus, a nucleotide sequence which is reverse complementary to the universal tag, a nucleotide sequence which is reverse complementary to a target molecule or a portion of a target molecule, and a fragment of poly (T) or poly (A) optionally added on the 5′ terminus.

2. The method according to claim 1, characterized in that the solid phase support is glass slide, plastic substrate, microbead or polymer membrane.

3. The method according to claim 1, characterized in that the solid phase support is modified with epoxy group, amine, poly-L-lysine, aldehyde group, carboxyl, or thiol.

4. The method according to claim 1, wherein the universal tag has a sequence selected from AGTGTCGTA, CAGGTCGCA, and AGGTCGCA.

5. The method according to claim 1, wherein the terminal group of the probe on the 3′ terminus or 5′ terminus is amine, thiol, carboxyl, or biotin.

6. The method according to claim 1, wherein the probe is linked to the solid phase support via the fragment of poly (T) or poly (A) added on the 3′ terminus or 5′ terminus.

7. A kit for multiple targets detection of biomolecules, comprising:
a universal tag, which is a fragment of DNA, RNA, peptide nucleic acid, or LNA, and is 3-20 mer in length;
probe arrays linked to a solid support; and
an optional instruction for using the kit;
wherein the probe arrays includes probes and the probe contains in order from 3′ terminus to 5′ terminus, a nucleotide sequence which is reverse complementary to a target molecule or a portion of a target molecule, and a nucleotide sequence which is reverse complementary to the universal tag, and a fragment of poly (T) or poly (A) optionally added on the 3′ terminus; or the probe contains in order from 3′ terminus to 5′ terminus, a nucleotide sequence which is reverse complementary to the universal tag, a nucleotide sequence which is reverse complementary to a target molecule or a portion of a target molecule, and a fragment of poly (T) or poly (A) optionally added on the 5′ terminus.

8. The kit according to claim 7, characterized in that, the universal tag is labeled with indicators which are selected from the group consisting of fluorescent dye, quantum dot, nanogold, isotope, and/or biotin.

9. The kit according to claim 7, characterized in that the solid phase support is glass slide, plastic substrate, microbead or polymer membrane.

10. The kit according to claim 7, characterized in that the solid phase support is modified with epoxy group, amine, poly-L-lysine, aldehyde group, carboxyl, or thiol, and the probe links to the solid phase support via a terminal group of amine, thiol, carboxyl, or biotin on the 3′ terminus or 5′ terminus of the probe.

11. The kit according to claim 7, wherein the universal tag has a sequence selected from AGTGTCGTA, CAGGTCGCA, and AGGTCGCA.

12. The kit according to claim 7, wherein the probe is linked to the solid phase support via the fragment of poly (T) or poly (A) optionally added on the 3′ terminus or 5′ terminus.


SEQUENCE LISTING (24 sequences)

SEQ ID NO: 1 - DNA, 38 bp, Artificial Sequence
  Synthetic Primer; The 5′terminus is modified with amino group
ttttttttttttaaccgctggcaacaaagtacgacact

SEQ ID NO: 2 - DNA, 38 bp, Artificial Sequence
  Synthetic Primer; The 5′terminus is modified with amino group
ttttttttttttgtaggtaaggttcttcgtacgacact

SEQ ID NO: 3 - DNA, 37 bp, Artificial Sequence
  Synthetic Primer; The 5′terminus is modified with amino group
ttttttttttttgcgcccgtttccggactacgacact

SEQ ID NO: 4 - DNA, 38 bp, Artificial Sequence
  Synthetic Primer; The 5′terminus is modified with amino group
ttttttttttttagcaagcttctcgtccgtacgacact

SEQ ID NO: 5 - DNA, 39 bp, Artificial Sequence
  Synthetic Primer; The 5′terminus is modified with amino group
ttttttttttttgtttccaagtgttatccctacgacact

SEQ ID NO: 6 - DNA, 9 bp, Artificial Sequence
  Synthetic Primer; The 5′terminus is modified with FAM
agtgtcgta

SEQ ID NO: 7 - DNA, 41 bp, Artificial Sequence
  Synthetic Primer; The 5′terminus is modified with amino group
aaaaaaaaaatccacatggagttgctgttacatgcgacctg

SEQ ID NO: 8 - DNA, 41 bp, Artificial Sequence
  Synthetic Primer; The 5′terminus is modified with amino group
aaaaaaaaaacaaacaccattgtcacactccatgcgacctg

SEQ ID NO: 9 - DNA, 41 bp, Artificial Sequence
  Synthetic Primer; The 5′terminus is modified with amino group
aaaaaaaaaaacaaagttctgtagtgcactgatgcgacctg

SEQ ID NO: 10 - DNA, 40 bp, Artificial Sequence
  Synthetic Primer; The 5′terminus is modified with amino group
aaaaaaaaaaggctgtcaattcataggtcagtgcgacctg

SEQ ID NO: 11 - DNA, 9 bp, Artificial Sequence
  Synthetic Primer; The 5′terminus is modified with nano gold
caggtcgca

SEQ ID NO: 12 - DNA, 39 bp, Artificial Sequence
  Synthetic Primer; The 5′terminus is modified with amino group
ttttttttttcagcatcggaccggtaatcgtacgacact

SEQ ID NO: 13 - DNA, 39 bp, Artificial Sequence
  Synthetic Primer; The 5′terminus is modified with amino group
tttttttttttgcgatcgcagcggtaaccttacgacact

SEQ ID NO: 14 - DNA, 39 bp, Artificial Sequence
  Synthetic Primer; The 5′terminus is modified with amino group
ttttttttttgaccatagtgcgggtaggtatacgacact

SEQ ID NO: 15 - DNA, 20 bp, Artificial Sequence
  Synthetic Primer
cgattaccggtccgatgctg

SEQ ID NO: 16 - DNA, 20 bp, Artificial Sequence
  Synthetic Primer
aggttaccgctgcgatcgca

SEQ ID NO: 17 - DNA, 20 bp, Artificial Sequence
  Synthetic Primer
tacctacccgcactatggtc

SEQ ID NO: 18 - DNA, 9 bp, Artificial Sequence
  Synthetic Primer; The 5′terminus is modified with FAM
agtgtcgta

SEQ ID NO: 19 - DNA, 40 bp, Artificial Sequence
  Synthetic Primer
aaaaaaaaaaaaccacacaacctactacctcatgcgacct

SEQ ID NO: 20 - DNA, 40 bp, Artificial Sequence
  Synthetic Primer
aaaaaaaaaaaactatacaacctactacctcatgcgacct

SEQ ID NO: 21 - DNA, 40 bp, Artificial Sequence
  Synthetic Primer
aaaaaaaaaaaactatacaatctactacctcatgcgacct

SEQ ID NO: 22 - DNA, 40 bp, Artificial Sequence
  Synthetic Primer
aaaaaaaaaaaactatgcaacctactacctcttgcgacct

SEQ ID NO: 23 - DNA, 8 bp, Artificial Sequence
  Synthetic Primer
aggtcgca

SEQ ID NO: 24 - RNA, 22 nt, Artificial Sequence
  Synthetic RNA
ugagguaguagguugugugguu